(19) United States
(12) Patent Application Publication
(10) Pub. No.: US 2023/0059586 A1
(43) Pub. Date: Feb. 23, 2023

(54) COOLING EQUIPMENT AND METHOD FOR PRODUCING COOLING EQUIPMENT

(71) Applicant: NIPPON TELEGRAPH AND TELEPHONE CORPORATION, Tokyo (JP)

(72) Inventors: Shingo TSUKADA, Musashino-shi, Tokyo (JP); Hiroshi NAKASHIMA, Musashino-shi, Tokyo (JP); Masumi YAMAGUCHI, Musashino-shi, Tokyo (JP); Takayuki OGASAWARA, Musashino-shi, Tokyo (JP); Kentaro TANAKA, Musashino-shi, Tokyo (JP); Toichiro GOTO, Musashino-shi, Tokyo (JP)

(73) Assignee: NIPPON TELEGRAPH AND TELEPHONE CORPORATION, Tokyo (JP)

(21) Appl. No.: 17/796,867

(22) PCT Filed: Feb. 7, 2020
(86) PCT No.: PCT/JP2020/004872

§ 371 Date: Aug. 1, 2022

(51) Int. Cl. A61F 7/10; A45F 3/18

(52) U.S. Cl. CPC A61F 7/103; A45F 3/18; A61F 2007/0098

(57) ABSTRACT

This cooling apparatus includes a tube (10) having an opening portion (10a) in one end portion in a longitudinal direction thereof, a cap (30) capable of opening/closing the opening portion (10a), a non-freezing drinkable coolant (20) that fills the tube (10), and a container (50) that contains the tube (10) filled with the coolant (20). The tube has a constricted portion (10d) having a reduced inner diameter.

### TECHNICAL FIELD

The present invention relates to a cooling apparatus and a method for manufacturing the cooling apparatus.

### BACKGROUND ART

Conventionally, cooling apparatuses to be worn by humans or animals to cool body surfaces of the humans or animals have been proposed.

For example, clothes for animals which protect domestic animals and pets from a cold stress and a temperature stress have been proposed. NPL 1 describes a bandanna to be wrapped around a neck of a dog which contains a refrigerant to be able to cool a body surface of the dog.

### CITATION LIST

### Non Patent Literature

[NPL 1] [online], 2020, [Retrieved on Jan. 8, 2020], Internet <URL: https://www.amazon.co.jp/dp/B076CZ1RZL/ref=psdc_2155279051_t2_B07DN6NMHV>

### SUMMARY OF THE INVENTION

### Technical Problem

Incidentally, in recent years, due to global warming and heat waves, an increasing number of people have been suffering heatstroke. In particular, in most cases, people suffer heatstroke during outdoor activities. To prevent a person participating in an outdoor activity from developing heatstroke, a cooling apparatus to be carried by the person to be able to continuously cool a body surface of the person is necessary. To prevent development of heatstroke, hydration as well as the cooling of the body surface is also necessary.

Even if the bandanna described in NPL 1 is applicable to a human, the bandanna has such a limited capacity as to allow the refrigerant to be wrapped around a neck of the human only once, and therefore the bandanna cannot continuously cool a body surface of the human. In addition, the bandanna described in NPL 1 has a problem in that a means for hydrating a user is not included therein.

In view of the circumstances described above, an object of the present invention is to provide a portable cooling apparatus capable of continuously cooling a body surface of a human body and providing hydration, and a method for manufacturing the cooling apparatus.

### Means for Solving the Problem

A cooling apparatus according to an aspect of the present invention includes: a tube having an opening portion only in one end portion in a longitudinal direction thereof; a cap capable of opening/closing the opening portion; a non-freezing drinkable coolant that fills the tube; and a container that contains the tube filled with the coolant, the tube having a constricted portion having a reduced inner diameter.

A method for manufacturing the cooling apparatus according to the aspect of the present invention includes: a filling step of filling the tube with the coolant in a fluid state; and a division step of applying, to the tube, a pressure perpendicular to the longitudinal direction of the tube to divide a range in which the coolant is present in the tube.

### Effects of the Invention

With the cooling apparatus and the method for manufacturing the cooling apparatus each described above, it is possible to provide a portable cooling apparatus capable of continuously cooling a body surface of a human body and providing hydration, and a method for manufacturing the cooling apparatus.

### BRIEF DESCRIPTION OF DRAWINGS

FIG. 1 is a perspective view of a cooling apparatus according to a first embodiment of the present invention.

FIG. 2 is a perspective view of a drinking spout of the cooling apparatus according to the first embodiment of the present invention.

FIG. 3 is a perspective view of the cooling apparatus according to the first embodiment of the present invention.

FIG. 4 is a diagram of the cooling apparatus according to the first embodiment of the present invention when used.

FIG. 5 is a diagram of the cooling apparatus according to the first embodiment of the present invention when used.

FIG. 6 is a diagram of a modification of the cooling apparatus according to the first embodiment of the present invention when used.

FIG. 7 is a diagram of another modification of the cooling apparatus according to the first embodiment of the present invention when used.

FIG. 8 is a diagram of the other modification of the cooling apparatus according to the first embodiment of the present invention when used.

FIG. 9 is a diagram of still another modification of the cooling apparatus according to the first embodiment of the present invention when used.

FIG. 10 is a diagram of a cooling apparatus according to a second embodiment of the present invention when used.

FIG. 11 is a diagram of a cooling apparatus according to a third embodiment of the present invention when used.

FIG. 12 is a cross-sectional view of a modification of a connecting portion of a tube according to an embodiment of the present invention.

FIG. 13 is a diagram illustrating a modification of a cooling apparatus according to the embodiment of the present invention.

FIG. 14 is a diagram illustrating a method for manufacturing the cooling apparatus according to the embodiment of the present invention.

FIG. 15 is a diagram illustrating a modification of the cooling apparatus according to the embodiment of the present invention.

### DESCRIPTION OF EMBODIMENTS

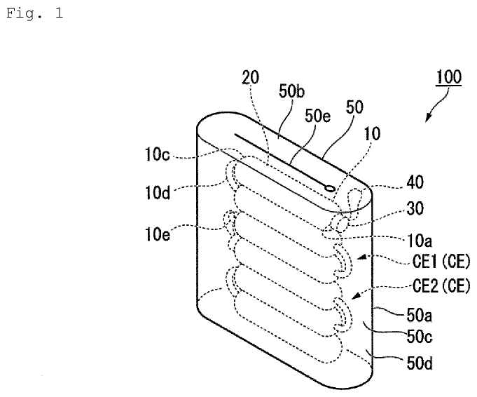

Referring to FIGS. 1 to 9, a description will be given of a first embodiment of the present invention.

A cooling apparatus 100 according to the present embodiment is a portable cooling apparatus which allows a user to drink a coolant 20 therefrom and receive hydration, while cooling a body surface of the user with the coolant 20.

FIG. 1 is a perspective view of the cooling apparatus 100 according to the first embodiment of the present invention. As illustrated in FIG. 1, the cooling apparatus 100 includes cooling equipment CE and a container 50. The cooling equipment CE includes a tube 10, the coolant 20, a cap 30, and an extraction handle 40. In the present embodiment, the cooling equipment CE includes first cooling equipment CE1 and second cooling equipment CE2 each having the same configuration and connected to each other.

The tube 10 is formed of a resin material such as, e.g., polyethylene, polypropylene, polyvinyl alcohol, nitrile rubber, or silicone. The resin material is not limited to those mentioned above, and a polymer material and an elastomer each forming a waterproof membrane can extensively be used. The tube 10 is elongated. FIG. 1 illustrates a state where the tube 10 is folded in a longitudinal direction.

The tube 10 has an opening portion 10a, coolant containing portions 10c, constricted portions 10d, and a hook 10e.

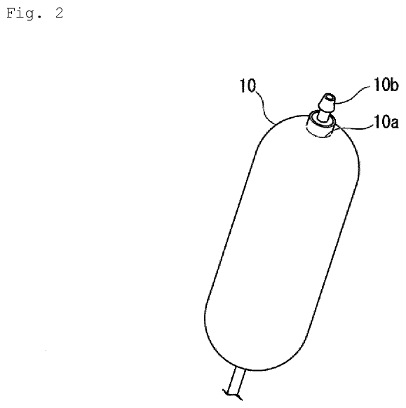

The opening portion 10a of the tube 10 is provided in one end portion of the tube 10 in the longitudinal direction. The opening portion 10a of the tube 10 can be opened/closed by the cap 30. FIG. 2 is a diagram illustrating the opening portion 10a of the tube 10 in a state where the cap 30 is detached. As illustrated in FIG. 2, the opening portion 10a is provided with a drinking spout 10b. The drinking spout 10b has a known check valve structure. The drinking spout 10b has a screw formed in, e.g., an outer surface thereof to be removably screwed onto the tube 10. The drinking spout 10b may also be fit into the tube to open/close the tube. The drinking spout 10b may also have an uneven shape to be engaged with the tube and open/close the tube.

As illustrated in FIG. 1, the tube 10 is provided with the coolant containing portions 10c and the constricted portions 10d which alternate along the longitudinal direction. The tube 10 has the coolant containing portions 10c at both ends thereof in the longitudinal direction.

The coolant containing portions 10c contain and hold the coolant 20. Each of the constricted portions 10d has an inner diameter smaller than an inner diameter of each of the coolant containing portions 10c. The constricted portions 10d allow the coolant 20 in a fluid state to flow between the longitudinally adjacent coolant containing portions 10c.

The hook 10e is provided at another end portion of the tube 10 which is other than the longitudinal end portion thereof provided with the opening portion 10a. The hook 10e can be engaged with the extraction handle 40. In FIG. 1, the hook 10e of the first cooling equipment CE1 is engaged with the extraction handle 40 of the second cooling equipment CE2.

The coolant 20 fills the tube 10. The coolant 20 is a drinkable substance that exhibits fluidity at a temperature in an environment in which a user H participates in an activity. As the coolant 20, e.g., water, salt water, a sweetened liquid, a liquid or a jelly containing a nutrient or the like, an oral rehydration solution, an electrolyte solution, or the like can be used. When the user H wears the cooling apparatus 100, the coolant 20 is set at a temperature lower than a temperature of a body surface of the user H to be brought in to, e.g., a frozen discrete state.

The cap 30 opens/closes the opening portion 10a of the tube 10. For example, the cap 30 has a cuplike shape, has a screw formed in an inner surface thereof, and removably screwed onto the tube 10. The cap 30 may also fit into the tube to open/close the tube. The cap 30 may also have an uneven shape to be engaged with the tube and open/close the tube.

The extraction handle 40 has a string shape and both ends attached to the cap 30. The extraction handle may also be attached to the coolant containing portion 10c to which the cap 30 is attached, not to the cap 30.

The container 50 contains the cooling equipment CE. The container 50 includes a container portion 50a and a lid portion 50b.

The container portion 50a has a boxlike shape that is open to one side. The container portion 50a includes an outer housing portion 50c and a heat insulating portion 50d. The outer housing portion 50c forms an outer surface of the container portion 50a. For the outer housing portion 50c, e.g., fabric, polyester, or the like can be used. The heat insulating portion 50d forms an inner surface of the container portion 50a. For the heat insulating portion 50d, a heat insulating material such as, e.g., aluminum foil, polyurethane foam, filling cotton, or the like can be used.

The lid portion 50b covers an opening of the container portion 50a. For the lid portion 50b, e.g., fabric, polyester, or the like can be used. The lid portion 50b has an edge joined to an end portion of the opening of the container portion 50a. The lid portion 50b has a slit 50e.

Next, a description will be given of an operation of the cooling apparatus 100 when used. The user H first takes out the cooling equipment CE from the container 50. The user H inserts his or her hand into the container portion 50a through the slit 50e. For example, the user H grasps the extraction handle 40 and takes out the first cooling equipment CE1 from the container 50 through the slit 50e.

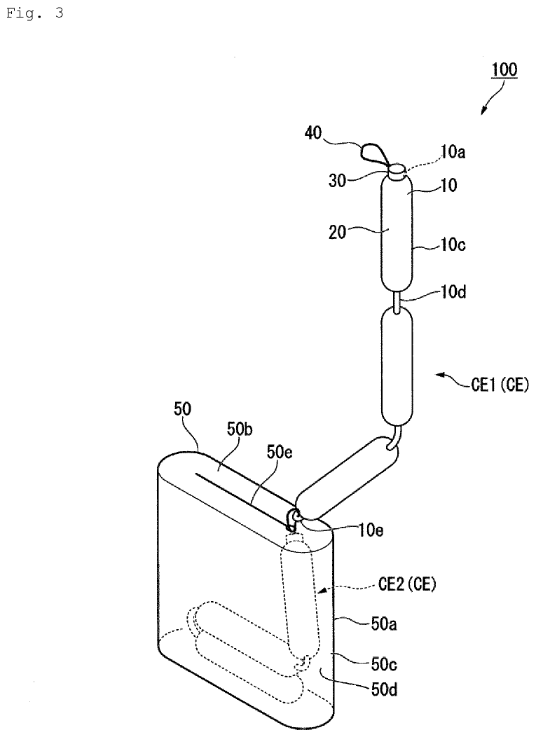

FIG. 3 illustrates a state where the first cooling equipment CE1 is taken out of the container 50. As illustrated in FIG. 3, when used, the first cooling equipment CE1 is taken out of the container 50, and the tube 10 is unfolded.

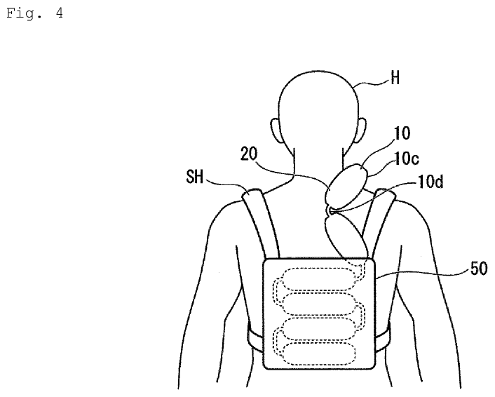

FIG. 4 is a diagram illustrating a back region of the user H in a state where the user H is drinking the coolant 20, while applying the tube 10 to the body surface. As illustrated in FIG. 4, the user H carries the container 50 via, e.g., a shoulder harness SH.

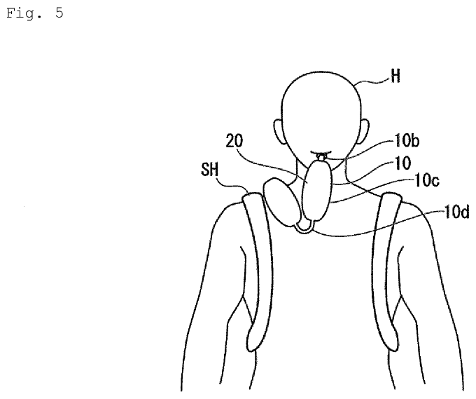

FIG. 5 is a diagram illustrating a front side of the user H in a state where the user H is drinking the coolant 20, while applying the tube 10 to the body surface. As illustrated in FIG. 5, the user H removes the cap 30 from the tube 10 and holds the drinking spout 10b in his or her mouth. The user H drinks the coolant 20 having absorbed heat from the body of the user H and reached a fluid state at an appropriate time.

When the coolant 20 in a portion of the first cooling equipment CE1 which is in contact with the body surface of the user H is lost as a result of drinking or the like, the user H further pulls out the first cooling equipment CE1 from the container 50. Meanwhile, when the coolant 20 in the portion of the first cooling equipment CE1 which is in contact with the body surface of the user H reaches a temperature equal to a body temperature of the user H as a result of absorbing heat, the user H further pulls out the first cooling equipment CE1 from the container 50.

Thus, the user H further pulls out the first cooling equipment CE1 from the container 50 when the coolant 20 in the portion of the first cooling equipment CE1 which is in contact with the body surface of the user H is lost or has undergone a temperature rise. The user H applies the portion of the first cooling equipment CE1 holding the coolant 20 at a temperature lower than the body temperature of the user H to the body surface.

When having completely consumed the coolant 20 in the first cooling equipment CE1 or the like, the user H further pulls out the first cooling equipment CE1 from the container 50. By further pulling out the first cooling equipment CE1 from the container 50, the user H takes out the second cooling equipment CE2 connected to the first cooling equipment CE1. The user H uses the second cooling equipment CE2 to continue to cool his or her body surface and drink the coolant 20.

The cooling apparatus 100 allows the user H to carry the tube 10 filled with the coolant 20 and allows the user H to cool his or her body surface by applying the tube 10 thereto. The user H can receive a water supply and a salt supply by drinking the coolant 20 from the drinking spout 10b provided in the tube 10.

In the tube 10, each of the constricted portions 10d has the inner diameter smaller than the inner diameter of each of the coolant containing portions 10c. Accordingly, even when the frozen coolant 20 is present in the constricted portion 10d, the user H can break the frozen coolant 20 and fold up the tube 10 or unfold the tube 10 at the constricted portion 10d. Even when the coolant 20 is a substance which freezes at a temperature equal to or lower than a normal temperature of the user H, the user H can fold up the tube 10 or unfold the tube 10. This allows the tube 10 to extend along the body surface of the user H. Even when the coolant 20 is lost from the coolant containing portion 10c of the tube 10 which is in contact with the body surface of the user H as a result of melting and drinking of the coolant 20, by pulling out another of the coolant containing portions 10c from the container 50, the user H can continue to cool the body surface.

Since the heat insulating portion 50d forms the inner surface of the container 50 containing the tube 10, it is possible to inhibit the tube 10 from absorbing heat from a place other than the body surface of the user H.

Since the tube 10 has the hook 10e engageable with the extraction handle 40, a plurality of the tubes 10 can be joined together.

Since the drinking spout 10b of the tube 10 has the check valve structure, even when the user H temporarily releases the drinking spout 10b, no air enters the tube 10. As a result, when the user H holds the drinking spout 10b again in his or her mouth and attempts to drink the coolant 20, the user H is prevented from sucking in air.

The tube 10 is taken out of the container 50 via the slit 50e. Accordingly, when the tube 10 is taken out of the container 50, formation of an opening in the container 50 is inhibited, and a temperature rise inside the container 50 is inhibited.

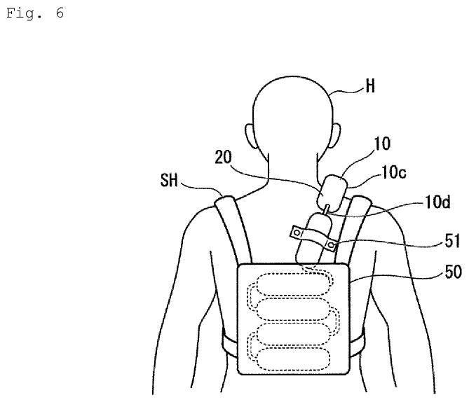

FIG. 6 is a diagram illustrating a modification of the cooling apparatus 100. As illustrated in FIG. 6, the cooling apparatus 100 may also include a strap (guide member) 51 serving as a guide member that guides placement of the tube 10 on the body surface of the user H. The strap 51 is formed in a band shape and has both ends thereof attached to clothes (not shown) of the user H to be worn by the user H. The strap 51 allows the tube 10 to extend between the strap 51 and the clothes and guides the placement of the tube 10 on the body surface of the user H.

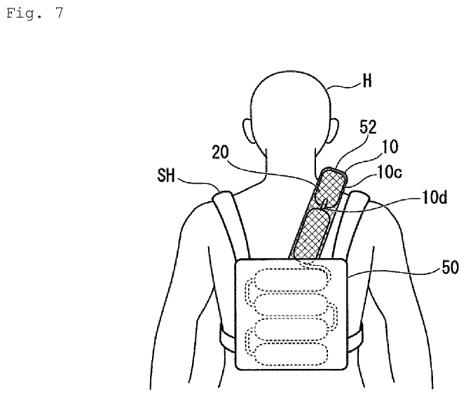

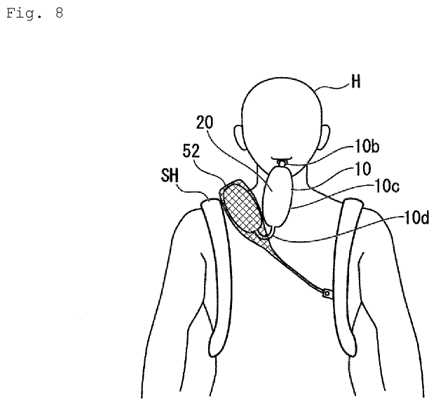

FIGS. 7 and 8 are diagrams illustrating a modification of the cooling apparatus 100. As illustrated in FIGS. 7 and 8, the cooling apparatus 100 may also include a mesh-shaped cylindrical band (guide member) 52. The band 52 is provided to, e.g., extend from the container 50 to the shoulder harness SH along the body surface of the user H to be worn by the user H. The band 52 allows the tube 10 to extend through the inside thereof and guides placement of the tube 10 on the body surface of the user H.

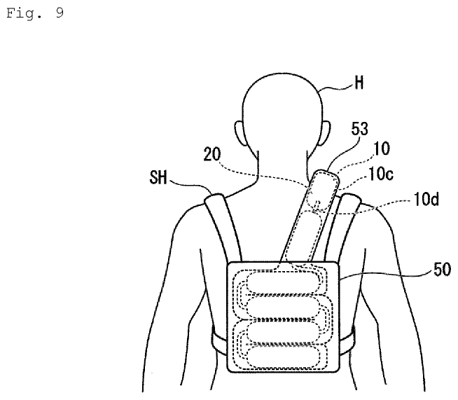

FIG. 9 is a diagram illustrating a modification of the cooling apparatus 100. As illustrated in FIG. 9, the cooling apparatus 100 may also include a covering member 53 that covers the tube 10. The covering member 53 is formed of, e.g., fabric or the like. The covering member 53 covers an outer peripheral surface of the tube 10 filled with the coolant 20 to inhibit the tube 10 from coming into direct contact with the body surface of the user H and giving a sense of discomfort to the user H. The covering member 53 may also include a heat insulating material on an inner peripheral surface thereof which is kept from contact with the body surface of the user H.

It may also be possible that the container does not have the slit 50e, and the opening of the container portion 50a is covered with a tab.

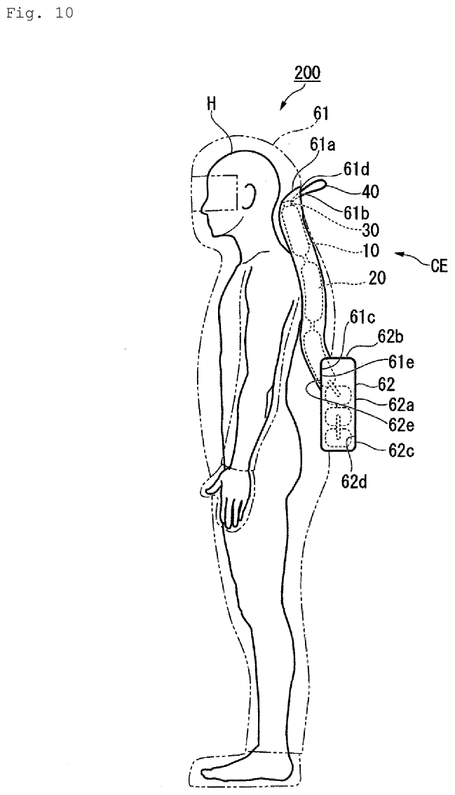

Next, referring to FIG. 10, a description will be given of a second embodiment of the present invention.

A cooling apparatus 200 according to the present embodiment includes a full-bodied suit 61 to be worn by the user H so as to cover the entire body thereof, such as, e.g., protective clothing or a mascot costume. The cooling apparatus 200 cools the body surface of the user H with the coolant 20.

As illustrated in FIG. 10, the cooling apparatus 200 includes the cooling equipment CE, the full-bodied suit 61, and a container 62. The cooling equipment CE has the same configuration as that of the cooling equipment CE of the cooling apparatus 100.

The full-bodied suit 61 is worn so as to cover the entire body of the user H and has a protective function such as chemical protection or a design in the form of a character.

The full-bodied suit 61 has a cooling equipment insertion portion 61a, a pull-out slit 61b, and an insertion slit 61c.

The cooling equipment insertion portion 61a is provided inside the full-bodied suit 61. The cooling equipment insertion portion 61a is formed into a cylindrical shape by using fabric, a sheet material, or the like. Into the cooling equipment insertion portion 61a, the tube 10 of the cooling equipment CE can be drawn. For the cooling equipment insertion portion 61a, a material having excellent airtightness or a high thermal conductivity may also be used. The cooling equipment insertion portion 61a has one opening portion 61d and another opening portion 61e each joined to an inner surface of the full-bodied suit 61.

The pull-out slit 61b is formed in a range of the full-bodied suit 61 which is surrounded by a joint portion between the full-bodied suit 61 and the opening portion 61d. The insertion slit 61c is formed in a range of the full-bodied suit 61 which is surrounded by a joint potion between the full-bodied suit 61 and the opening portion 61e.

The container 62 contains the cooling equipment CE. The container 62 includes a container portion 62a and a lid portion 62b.

The container portion 62a has a boxlike shape that is open to one side. The container portion 62a is connected to the full-bodied suit 61 so as to face the insertion slit 61c of the full-bodied suit 61. The container portion 62a includes an outer housing portion 62c, a heat insulating portion 62d, and a slit 62e.

The outer housing portion 62c forms an outer surface of the container portion 62a. For the outer housing portion 62c, e.g., fabric, polyester, or the like can be used. The heat insulating portion 62d forms an inner surface of the container portion 62a. For the heat insulating portion 62d, e.g., aluminum foil or the like can be used. The slit 62e is formed in the container portion 62a so as to face the insertion slit 61c of the full-bodied suit 61.

The lid portion 62b opens/closes an opening of the container portion 62a. For the lid portion 62b, e.g., fabric, polyester, or the like can be used. The lid portion 62b has an edge connected to the opening of the container portion 62a by using, e.g., a fastener.

When the cooling apparatus 200 is used, first, the user H opens the lid portion 62b of the container 62 to allow the cooling equipment CE to be contained in the container 62.

The user H inserts the tube 10 of the cooling equipment CE from the container 62 into the cooling equipment insertion portion 61a via the slit 62e and the insertion slit 61c. The user H inserts the tube 10 into the cooling equipment insertion portion 61a such that the inserted tube 10 extends over an entire length thereof.

The user H closes the lid portion 62b of the container 62. The user H wears the full-bodied suit 61.

The cooling apparatus 200 allows the user H who wears the full-bodied suit 61 and participates in an activity to carry the tube 10 filled with the coolant 20 and cool the body surface of the user H by applying the tube 10 thereto via the cooling equipment insertion portion 61a.

The cooling apparatus 200 allows the cooling equipment CE to be pulled out of the pull-out slit 61b. The lid portion 62b of the container 62 externally connected to the full-bodied suit 61 is openable/closable. The cooling equipment CE can be inserted from the container 62 into the cooling equipment insertion portion 61a via the slit 62e and the insertion slit 61c. Consequently, the cooling equipment CE having reached a temperature in an environment in which the user H is participating in the activity is pulled out of the pull-out slit 61b to be discarded. After the cooling equipment CE having reached the temperature in the environment in which the user H is participating in the activity is discarded, a cooperating worker or the like can cause the container 62 to contain the cooling equipment CE again and insert the tube 10 into the cooling equipment insertion portion 61a. As a result, the user H wearing the full-bodied suit 61 and participating in the activity can continue to cool the body surface. The user H can place the cooling equipment CE having reached the temperature in the environment in which the user H is participating in the activity in the full-bodied suit 61 and receive hydration by drinking the coolant 20.

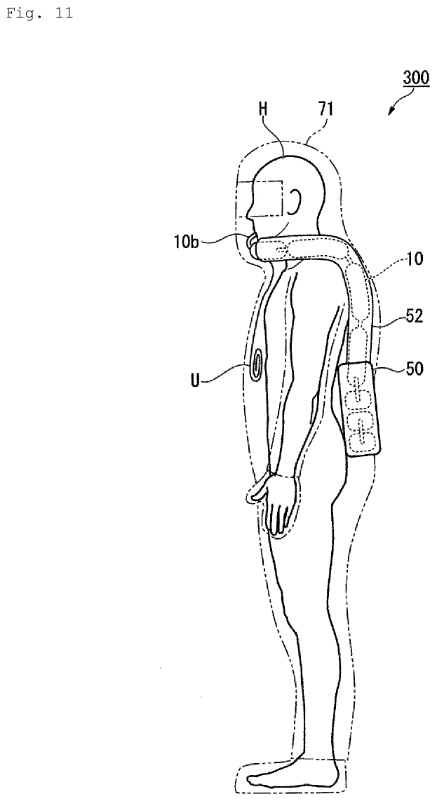

Next, referring to FIG. 11, a description will be given of a third embodiment of the present invention.

A cooling apparatus 300 according to the present embodiment includes a full-bodied suit 71 to be worn by the user H so as to cover the entire body thereof, such as, e.g., protective clothing or a mascot costume. The cooling apparatus 300 is a cooling apparatus that allows the user H to drink the coolant 20 and receive hydration, while cooling the body surface of the user H with the coolant 20.

The cooling apparatus 300 includes the full-bodied suit 71, the container 50, the band 52, and the tube 10.

Unlike the full-bodied suit 61 of the cooling apparatus 200, the full-bodied suit 71 has none of the cooling equipment insertion portion 61a, the pull-out slit 61b, and the insertion slit 61c. Into the full-bodied suit 71, the tube 10 including the container 50 is withdrawn.

The container 50 is placed inside the full-bodied suit 71. The band 52 is placed to extend along, e.g., an inner surface of the full-bodied suit 71. The tube 10 is contained in the container 50 to extend through the band 52 and have the drinking spout 10b held in the mouth of the user H.

The cooling apparatus 300 allows the user H who wears the full-bodied suit 71 and participates in an activity to carry the tube 10 filled with the coolant 20 and cool the body surface of the user H by applying the tube 10 thereto via the band 52. The user H can drink the coolant 20 from the drinking spout 10b provided in the tube 10 and receive hydration.

While each of the embodiments of the present invention has been described in detail with reference to the drawings, specific configurations are not limited to the embodiment, and design changes and the like may also be made without departing from the gist of the present invention. In addition, the components described in the embodiments described above and a modification described below can be configured by being appropriately combined.

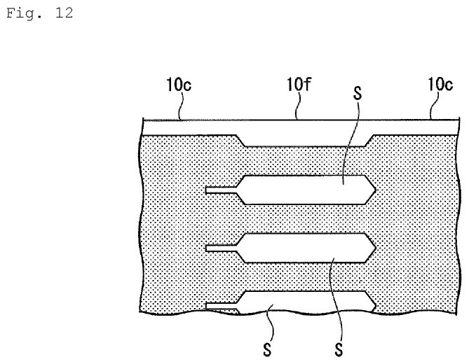

As illustrated in FIG. 12, the constricted portions of the tube may also be such that, instead of reducing outer and inner diameters thereof rather than the coolant containing portions 10c, the inner diameters thereof are reduced by e.g., thermally welding inner surface portions of the tube to each other and thereby forming sealed portions S.

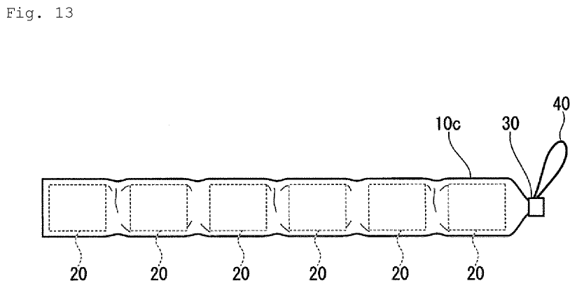

As illustrated in FIG. 13, it may also be possible that the tube does not have the constricted portions 10d, and is filled with a plurality of the coolants 20 frozen and formed in block shapes.

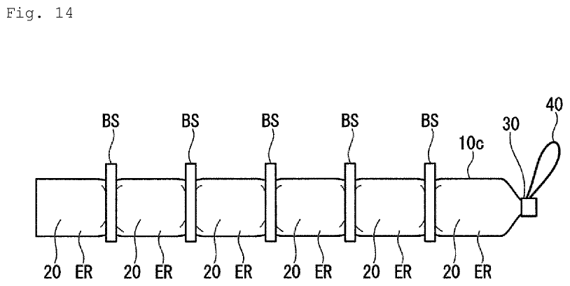

To fill the tube with a plurality of the coolants 20 frozen and formed in the block shapes, first, the tube is filled with the coolant 20 in a fluid state (filling step). Then, as illustrated in FIG. 14, the tube is clipped with board sealers BS or the like along an entire length of the tube in a lateral direction thereof. As a result, pressures perpendicular to a longitudinal direction of the tube are applied to the tube to divide a range ER in which the coolant 20 is present in the tube (division step). Then, the tube filled with the coolant 20 and having the divided range ER in which the coolant 20 is present is cooled to a temperature equal to or less than the normal temperature of a human (cooling step).

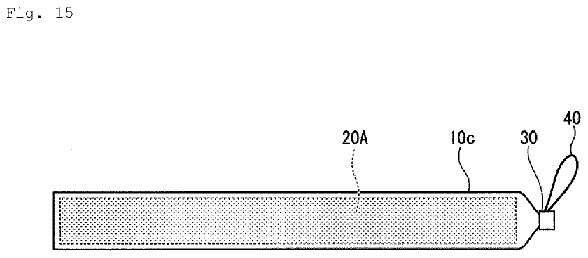

As illustrated in FIG. 15, it may also be possible that the tube does not have the constricted portions 10d, and is filled with a non-freezing coolant 20A.

The end portion of the tube longitudinally opposite to the end portion thereof formed with the opening portion 10a may also be configured to be openable/closable and allow filling of the tube with the coolant, removal of air from inside the tube, or cleaning of the inside of the tube to be promptly performed.

### REFERENCE SIGNS LIST

100, 200, 300 Cooling apparatus
10 Tube
10a Opening portion
10b Drinking spout
10c Coolant containing portion
10d Constricted portion
10e Hook
20, 20A Coolant
30 Cap
40 Extraction handle
50, 62 Container
50a, 62a Container portion
50b, 62b Lid portion
50c, 62c Outer housing portion
50d, 62d Heat insulating portion
50e, 62e Slit
51 Strap (Guide member)
52 Band (Guide member)
53 Covering member
61, 71 Full-bodied suit
61a Cooling equipment insertion portion
CE Cooling equipment
H User
SH Shoulder harness

1. A cooling apparatus comprising:
a tube having an opening portion in one end portion in a longitudinal direction thereof;
a cap capable of opening/closing the opening portion;
a non-freezing drinkable coolant that fills the tube; and
a container that contains the tube filled with the coolant, the tube having a constricted portion having a reduced inner diameter.

2. The cooling apparatus according to claim 1, wherein
the container has a heat insulating portion at an inner surface thereof.

3. The cooling apparatus according to claim 1, which has a covering member that covers an outer peripheral surface of the tube.

4. The cooling apparatus according to claim 1, which has a guide member that is wearable by a human and guides the tube from the container.

5. The cooling apparatus according to claim 1, further comprising:
a full-bodied suit to which the container is attached and which allows the tube to be withdrawn therein.

6. The cooling apparatus according to claim 1, wherein the tube includes a drinking spout having a check function in the opening portion.

7. A method for manufacturing the cooling apparatus according to claim 1, the method comprising:
a filling step of filling the tube with the coolant in a fluid state; and
a division step of applying, to the tube, a pressure perpendicular to the longitudinal direction of the tube to divide a range in which the coolant is present in the tube.
